(19) United States
(12) Patent Application Publication
(10) Pub. No.: US 2025/0232757 A1
(43) Pub. Date: Jul. 17, 2025

(54) CONTROL DEVICE

(71) Applicant: TOYOTA JIDOSHA KABUSHIKI KAISHA, Toyota-shi (JP)

(72) Inventors: Takahiro UCHIDA, Kasugai-shi (JP); Seiji ARAKAWA, Nisshin-shi (JP); Kazuhito SAKAI, Mishima-shi (JP); Chie FUKUHARA, Toyota-shi (JP)

(73) Assignee: TOYOTA JIDOSHA KABUSHIKI KAISHA, Toyota-shi (JP)

(21) Appl. No.: 18/926,844

(22) Filed: Oct. 25, 2024

(30) Foreign Application Priority Data
Jan. 15, 2024 (JP) 2024-003968

(51) Int. Cl. G10K 15/02; A61B 5/024

(52) U.S. Cl. CPC G10K 15/02; A61B 5/02405

(57) ABSTRACT

The control device acquires biometric information of the driver by using a sensor mounted on the driver or a sensor mounted on battery electric vehicle. Further, the control device performs tone control for changing the tone of the pseudo engine sound based on the information of the preference of the driver or the biometric information.

### CROSS-REFERENCE TO RELATED APPLICATION

This application claims priority to Japanese Patent Application No. 2024-003968 filed on Jan. 15, 2024, incorporated herein by reference in its entirety.

### BACKGROUND

### 1. Technical Field

The present disclosure relates to a control device mounted on a battery electric vehicle that uses an electric motor as a power unit for travel.

### 2. Description of Related Art

Japanese Unexamined Patent Application Publication No. 2011-215437 (JP 2011-215437 A) discloses a sound control device provided in a vehicle that can be driven by an electric motor. The sound control device adds a sound effect corresponding to a result of simulating the operation of a virtual vehicle component to a virtual engine sound (pseudo engine sound) controlled based on the engine speed.

### SUMMARY

It is assumed that some drivers of battery electric vehicles that simulate a manual transmission vehicle are not satisfied with the pseudo engine sound. In this case, the driver cannot enjoy driving the battery electric vehicle. There is desired a technique capable of providing a driver with a pseudo engine sound that further increases the excitement of the driver.

An aspect of the present disclosure relates to a control device mounted on a battery electric vehicle that uses an electric motor as a power unit for travel and has a manual transmission (MT) mode in which a manual transmission vehicle is simulated.

The control device includes: one or more storage devices that store preference information on preferences of a driver of the battery electric vehicle; and one or more processors configured to generate a pseudo engine sound and output the pseudo engine sound through one or more in-vehicle speakers.

The one or more processors are configured to acquire biometric information on the driver using a sensor mounted to the driver or a sensor mounted on the battery electric vehicle. The one or more processors are further configured to perform tone control for changing a tone of the pseudo engine sound based on the preference information or the biometric information.

According to the present disclosure, the tone of the pseudo engine sound changes based on the preference information on the driver or the biometric information on the driver acquired by the sensor. Thus, the excitement of the driver can be further increased. Consequently, the driver can enjoy driving the battery electric vehicle that simulates a manual transmission vehicle better.

### BRIEF DESCRIPTION OF THE DRAWINGS

Features, advantages, and technical and industrial significance of exemplary embodiments of the disclosure will be described below with reference to the accompanying drawings, in which like signs denote like elements, and wherein:

FIG. 1 is a conceptual diagram illustrating a battery electric vehicle and a control device;

FIG. 2 is a block diagram illustrating an example of a functional configuration of a control device;

FIG. 3A is a diagram for explaining an example of a specific example of tone control;

FIG. 3B is a diagram for explaining another example of a concrete example of articulation control;

FIG. 4A is a block-diagram illustrating an exemplary configuration of a power control system of a battery electric vehicle according to an embodiment of the present;

FIG. 4B is a block-diagram illustrating another example of a configuration of a power control system of a battery electric vehicle;

FIG. 5 is a diagram illustrating an engine model, a clutch model, and a transmission model constituting a MT vehicle model; and

FIG. 6 is a diagram illustrating a torque-characteristics of an electric motor realized by motor control using an MT vehicle-model.

### DETAILED DESCRIPTION OF EMBODIMENTS

Embodiments of the present disclosure will be described with reference to the accompanying drawings.

### 1. Overview

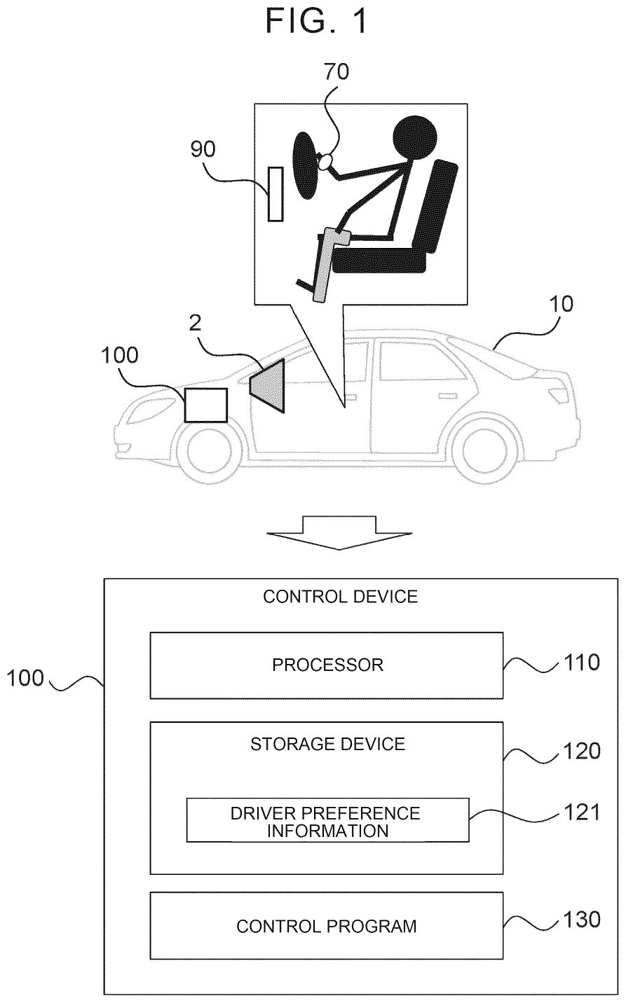

FIG. 1 is a conceptual diagram illustrating a battery electric vehicle 10 (hereinafter, simply referred to as a vehicle 10) and a control device 100 according to the present embodiment. The vehicle 10 uses an electric motor as a driving power device. The vehicle 10 also includes a MT modeling to simulate a manual transmission (MT) vehicle. Details of the configuration of the power control system of the vehicle 10 will be described later.

As illustrated in FIG. 1, the vehicle 10 includes one or a plurality of in-vehicle speakers 2 (hereinafter, simply referred to as speakers 2) and a control device 100. For example, the speaker 2 outputs sound to at least one of the inside and the outside of the vehicle. That is, the speaker 2 may include both an in-vehicle speaker and an out-vehicle speaker.

The control device 100 is connected to the speaker 2, generates a sound output from the speaker 2, and outputs the generated sound through the speaker 2. For example, the control device 100 generates a “pseudo engine sound” simulating the engine sound of the engine vehicle, and outputs the pseudo engine sound through the speaker 2.

Generally speaking, the control device 100 includes one or more processors 110 (hereinafter simply referred to as processors 110) and one or more storage devices 120 (hereinafter simply referred to as storage devices 120). The processor 110 executes various processes. Exemplary processors 110 include general purpose processors, special purpose processors, Central Processing Unit (CPU), Graphics Processing Unit (GPU), Application Specific Integrated Circuit (ASIC), Field-Programmable Gate Array (FPGA), integrated circuits, conventional circuits, and/or combinations thereof. The processor 110 may also be referred to as a circuitry or a processing circuitry. Circuitry is hardware programmed to implement the described functions, or hardware that performs the functions. The storage device 120 stores various types of information. Examples of the storage device 120 include volatile memory, non-volatile memory, Hard Disk Drive (HDD), Solid State Drive (SSD), and the like. The function of the control device 100 is realized by the cooperation of the processor 110 and the storage device 120.

The control program 130 is a computer program executed by the processor 110. The functions of the control device 100 may be realized by the processor 110 in cooperation with the processor 110 executing the control program 130 and the storage device 120. The control program 130 is stored in the storage device 120. Alternatively, the control program 130 may be recorded in a computer-readable recording medium.

Consider a case where the pseudo engine sound is output from the speaker 2 in the vehicle 10. In this case, the basic sound source data for generating the pseudo engine sound is prepared in the storage device 120. The basic sound source data includes, for example, sound source data of sound caused by engine combustion, sound source data of sound caused by a drive system such as a gear, sound source data of noise sound, sound source data of event sound, and the like. Sound source data of the sound caused by the engine combustion is for low rotation, medium rotation, and high rotation. Sound source data of sound caused by a drive system such as a gear may be used for low rotation, medium rotation, or high rotation. Further, the control device 100 acquires driving state information indicating the driving state of the vehicle 10. Examples of the driving state of the vehicle 10 include a driving operation amount by a driver, a rotation speed of a wheel, a speed, a virtual engine rotation speed, and the like. The driving state information is detected by a sensor mounted on the vehicle 10 or calculated based on a detection result. The virtual engine rotation speed is a rotation speed of the virtual engine when the vehicle 10 is assumed to be driven by the virtual engine. For example, the virtual engine rotational speed is calculated based on the rotational speed of the wheel, the overall reduction ratio, and the slip ratio of the virtual clutch. Then, the control device 100 generates a pseudo engine sound according to the operating state (e.g., virtual engine rotation speed) of the vehicle 10 by combining one or more pieces of fundamental sound source data. Furthermore, the control device 100 outputs the generated pseudo engine sound through the speaker 2. The method of generating the pseudo engine sound is not particularly limited. For example, a pseudo-engine sound may be generated by a well-known pseudo-engine sound simulator employed in a game or the like. In addition, the vehicle type of the engine vehicle, which is a target for simulating the engine sound, may be designated by the driver.

Consider a pseudo-engine sound for increasing the tension of a driver. In order to increase the driver's tension, it is desirable to provide the driver with a pseudo-engine sound that is preferred by the driver or a pseudo-engine sound that enhances the driver's heartbeat.

Therefore, according to the present embodiment, the tone control for changing the tone of the pseudo engine sound output from the speaker 2 is performed on the basis of the driver preference information 121 or the biometric information of the driver. The articulation indicates, for example, the articulation of a sound, the pitch of a sound, and the like. Details of the tone control will be described later.

The driver preference information 121 is information related to the driver preference and is obtained from the driver. Alternatively, the driver preference information 121 may be estimated based on information acquired from the driver. The driver preference information 121 includes, for example, information about the driver's preference sounds (e.g., favorite songs, favorite musical instrument sounds). The driver's preferred sound may be a sample of a portion of the sound.

The driver preference information 121 is stored in the storage device 120. More specifically, the vehicle 10 is equipped with Human-Machine Interface (HMI) 90. HMI 90 includes a communication device and an inputting device capable of communicating with the control device 100. Examples of the input device include a touch panel and buttons. The driver uses the input device to input information about the preference of the driver. In 20 this way, the control device 100 can acquire the driver preference information 121 through HMI 90 device.

The biometric information of the driver is information related to the biometric of the driver, and includes information on the heart rate of the driver and the like. The biometric information of the driver is acquired using the sensor 70. The sensor 70 includes a sensor mounted on a driver, a sensor mounted on the vehicle 10, and the like. The sensor attached to the driver is, for example, an electrocardiogramat is installed in a wearable terminal such as a smart watch and detects the heart rate of the driver. The sensor mounted on the vehicle 10 is, for example, an electrocardiogram installed in a device (e.g., a handle or a seat belt) used by the driver during a driving operation and detects a heart rate of the driver. The control device 100 may include a communication device capable of communicating with the sensor 70 so that detection information by the sensor 70 can be acquired.

The control device 100 (processor 110) generates a pseudo-engine sound and outputs the pseudo-engine sound through the speaker 2. Then, the control device 100 (the processor 110) acquires biometric information of the driver by using the sensor 70 mounted on the driver or the sensor 70 mounted on the vehicle 10. Further, the control device 100 (the processor 110) performs tone control for changing the tone of the pseudo engine sound based on the driver preference information 121 or the biometric information of the driver. Thus, the tension of the driver can be further increased. Consequently, the driver can enjoy driving battery electric vehicle simulating the manual transmission vehicle.

### 2. Functional Configuration Examples

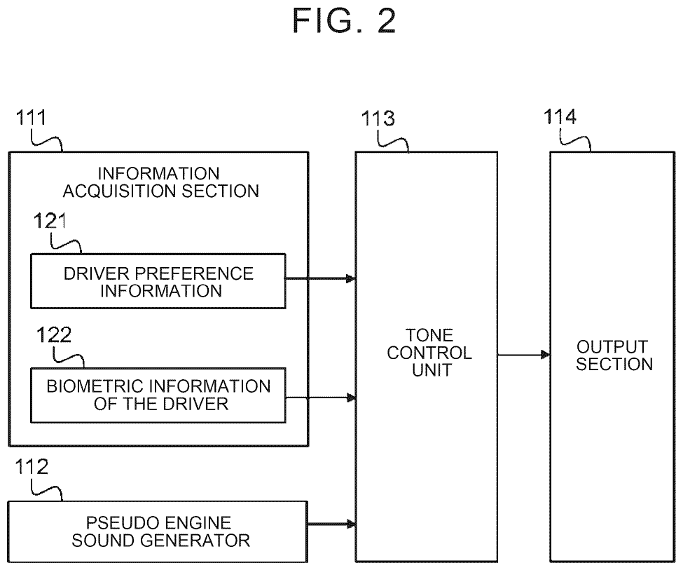

FIG. 2 is a block diagram illustrating an example of a functional configuration of the control device 100 according to the embodiment. The control device 100 includes, as functional blocks, an information acquisition unit 111, a pseudo engine sound generation unit 112, a tone control unit 113, and an output unit 114. These functional blocks may be realized by cooperation of the processor 110 executing the control program 130 and the storage device 120.

The information acquisition unit 111 reads the driver preference information 121 from the storage device 120. Alternatively, the information acquisition unit 111 acquires the biometric information 122 of the driver from the sensor 70. Then, the information acquisition unit 111 outputs the driver preference information 121 or the biometric information 122 of the driver to the tone control unit 113.

The pseudo engine sound generation unit 112 generates a pseudo engine sound. The pseudo-engine sound is generated, for example, by the method described above. Then, the pseudo engine sound generation unit 112 outputs the pseudo-engine sound to the tone control unit 113.

The tone control unit 113 performs tone control for changing the tone of the pseudo engine tone based on the driver preference information 121 or the biometric information 122 of the driver. Then, the tone control unit 113 outputs, to the output unit 114, the changed pseudo engine tone obtained by changing the tone of the pseudo engine tone. A specific example of the tone control will be described later.

The output unit 114 outputs the changed pseudo engine sound through the speaker 2.

### 3. Concrete Example of Tone Control

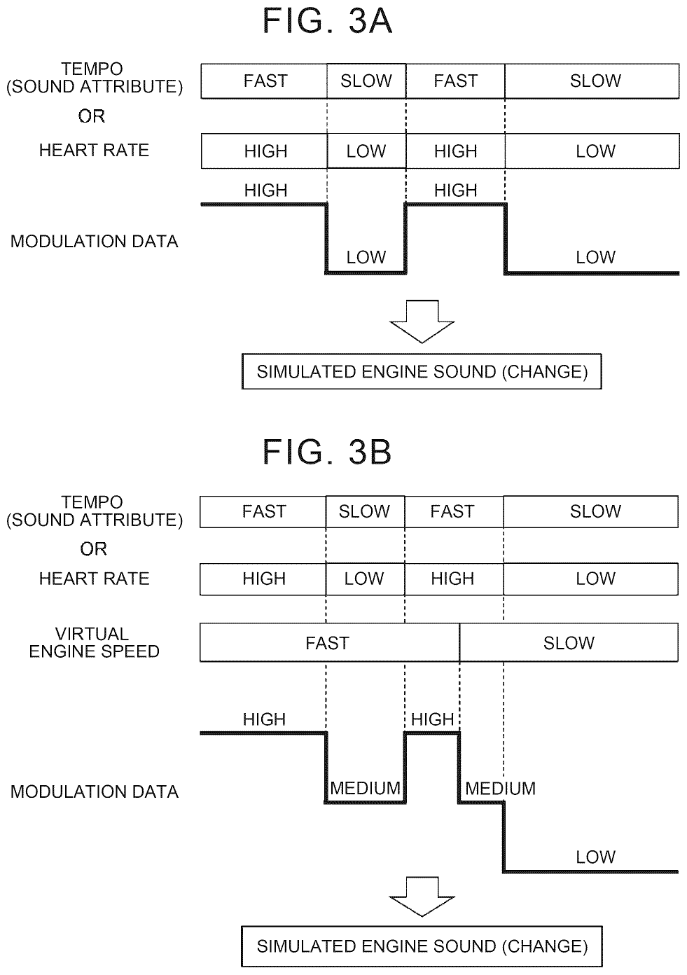

FIGS. 3A and 3B of the drawings are diagrams for explaining a specific embodiment of the tone control according to the embodiment. The control device 100 generates modulation data in the tone control. The modulation data is data used to change the tone of the pseudo engine sound. For example, the modulation data is generated based on the attribute of the driver's favorite sound included in the driver preference information 121 or the heart rate of the driver included in the biometric information 122 of the driver. The attribute of the sound is, for example, the tempo, rhythm, or the like of the sound (song). The attribute information indicating the attribute of the sound is associated with the favorite sound of the driver, and is included in the driver preference information 121.

For example, as shown in FIG. 3A, when the tempo of the sound that is the sound attribute is equal to or greater than the threshold value, the control device 100 determines that the tempo of the sound is “fast”. When the tempo of the sound is less than the threshold value, the control device 100 determines that the tempo of the sound is “slow”. Alternatively, when the heart rate is equal to or higher than the predetermined value, the control device 100 determines that the heart rate is “high”. When the heart rate is less than the predetermined value, the control device 100 determines that the heart rate is “low”. Then, the control device 100 generates the modulated data as “high” when the tempo of the sound desired by the driver is “fast” or the heart rate of the driver is “high”. If the driver's preferred sound tempo is “slow” or the driver's heart rate is “low”, the control device 100 generates the modulated data as “low”.

As another example, the modulation data may be further generated based on the operating state information (e.g., virtual engine speed) used to generate the pseudo engine sound described above. For example, as shown in FIG. 3B, when the virtual engine rotational speed is equal to or higher than the reference speed, the control device 100 determines that the speed is “fast”. When the virtual engine rotational speed is less than the reference speed, the control device 100 determines that the speed is “slow”. Then, the control device 100 generates modulation data as “high” when the tempo of the sound desired by the driver is “fast” or the heart rate of the driver is “high” and the virtual engine rotational speed is “fast”. If the driver's preferred sound tempo is “slow” or the driver's heart rate is “low” and the virtual engine speed is “fast”, the control device 100 generates the modulated data as “medium”. If the driver's preferred sound tempo is “fast” or the driver's heart rate is “high” and the virtual engine speed is “slow”, the control device 100 generates the modulated data as “medium”. If the driver's preferred sound tempo is “slow” or the driver's heart rate is “low” and the virtual engine speed is “slow”, the control device 100 generates the modulated data as “low”.

Note that the modulation data may be generated based on generation data obtained by multiplying one of the driver's favorite sound attributes (tempo, etc.) and the driver's heart rate by the virtual engine rotation speed. For example, when the generated data is equal to or larger than the reference value, the control device 100 may generate the modulated data as “high”. If the generated data is less than the reference value, the control device 100 may generate the modulated data as “low”.

Then, the control device 100 changes the pseudo engine sound based on the generated modulation data in the tone control. Specifically, the control device 100 adjusts the set value of the pseudo engine sound so as to change the pseudo engine sound in accordance with the modulation data. The set value of the pseudo engine sound is, for example, a sound pressure, a frequency, a sound range, or the like. The control device 100 adjusts at least one of the sound pressure, the frequency, and the sound range of the pseudo engine sound. Note that there are various methods for adjusting the set value of the pseudo engine sound. For example, when the modulation data is “high”, the sound pressure of the pseudo engine sound may be increased, the frequency of the pseudo engine sound may be increased, the sound range of the pseudo engine sound may be increased, or the frequency may be decreased so that the pseudo engine sound becomes a heavy bass sound. On the other hand, when the modulation data is “low”, for example, the sound pressure of the pseudo engine sound may be reduced, the frequency of the pseudo engine sound may be lowered, or the sound range of the pseudo engine sound may be lowered. When the modulation data is “medium”, for example, the setting value of the pseudo engine sound may be set to an initial value when the pseudo engine sound is generated. Note that the information on the setting value of the pseudo engine sound may be stored in the storage device 120.

According to the tone control, the tone of the pseudo engine tone can be changed in accordance with the attribute of the favorite tone of the driver. In addition, the articulation control can change the articulation of the pseudo engine sound in accordance with the variation of the heart rate. This can provide the driver with a pseudo-engine sound that is preferred by the driver or a pseudo-engine sound that enhances the heartbeat of the driver. Therefore, the tension of the driver can be further increased. Consequently, the driver can enjoy driving battery electric vehicle simulating the manual transmission vehicle.

### 4. Details about MT Modes

A typical battery electric vehicle does not include a manual transmission (MT) that switches the gear ratio by manual operation of a driver. On the other hand, an electric motor used as a driving power device in a battery electric vehicle can relatively easily control the torque by controlling an applied voltage and a field. Therefore, in the electric motor, it is possible to obtain a desired torque characteristic within the operating range of the electric motor by performing appropriate control. By taking advantage of this characteristic, the torque of battery electric vehicle can be controlled to simulate the torque characteristic peculiar to MT vehicles. A pseudo-shifter may also be provided on battery electric vehicle to allow the drivers to obtain driving sensations such as MT vehicles. As a result, MT vehicles can be simulated in battery electric vehicle.

In other words, battery electric vehicle controls the power of the electric motor so as to simulate MT vehicle-specific operating characteristics (torque-characteristics). The driver performs a pseudo manual shift operation by operating a pseudo shifter. Responsive to the simulated manual shifting by the drivers, battery electric vehicle simulates MT vehicles and changes the operating characteristics (torque-characteristics). This allows the drivers of battery electric vehicle to feel as if they are driving MT vehicles. The control mode of the electric motor for simulating the driving characteristic of MT vehicles and the manual shift operation is hereinafter referred to as a “manual mode” or a “MT mode”.

Hereinafter, a case will be considered in which the vehicle 10 according to the present disclosure is a battery electric vehicle 10E that is provided with MT mode. In MT mode, battery electric vehicle 10E may generate a pseudo-engine sound corresponding to the driving operation of the driver, and output the pseudo-engine sound via the speaker. Since not only driving maneuvers of MT vehicles but also engine-sounds of MT vehicles are reproduced, the degree of satisfaction of drivers seeking reality is increased. Hereinafter, an exemplary configuration of a battery electric vehicle 10E including MT mode will be described. Examples of MT modes include a “sequential shift mode” and a “three-pedal mode”.

### 4-1. First Configuration Example (Sequential Shift Mode)

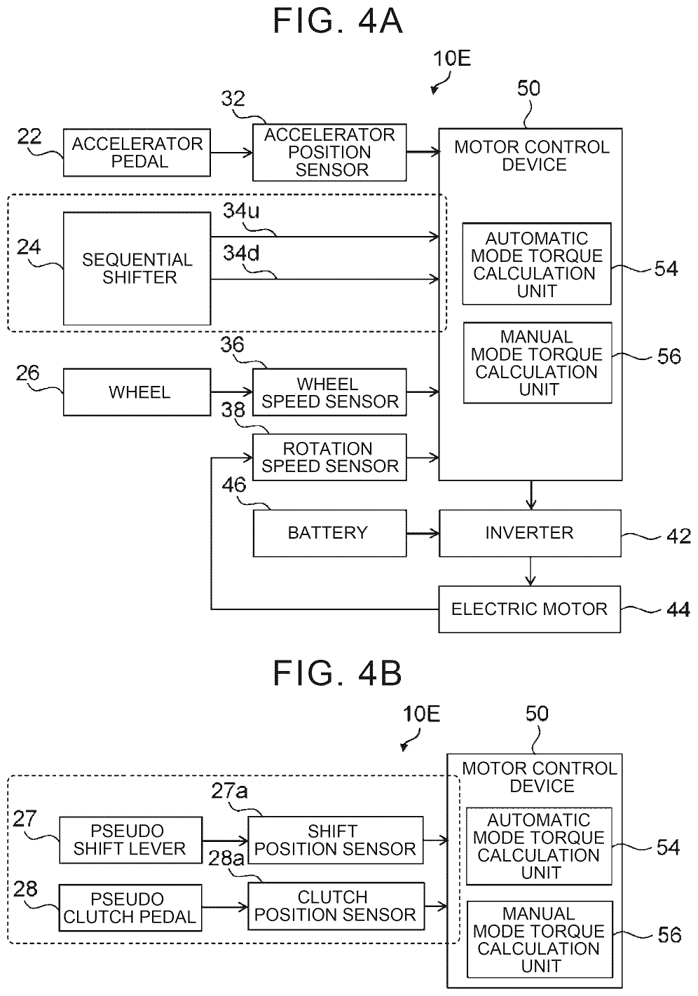

FIG. 4A is a block-diagram illustrating a first configuration of a power control system of a battery electric vehicle 10E. Battery electric vehicle 10E comprises an electric motor 44, a battery 46 and inverter 42. The electric motor 44 is a driving power device. The battery 46 stores electrical energy that drives the electric motor 44. That is, battery electric vehicle 10E is a battery electric vehicle (BEV that runs with the electric energy stored in the battery 46). The inverter 42 converts DC power input from the battery 46 into drive power of the electric motor 44 at the time of acceleration. In addition, the inverter 42 converts the regenerative power input from the electric motor 44 into DC power at the time of deceleration, and charges the battery 46.

Battery electric vehicle 10E comprises an accelerator pedal 22 for the driver to enter an acceleration demand for battery electric vehicle 10E. The accelerator pedal 22 is provided with an accelerator position sensor 32 for detecting an accelerator operation amount.

Battery electric vehicle 10E comprises a sequential shifter 24. The sequential shifter 24 may be a paddle type shifter or a lever type pseudo shifter.

The paddle shifter is a different dummy than the original paddle shifter. The paddle-type shifter has a configuration similar to a paddle-type shifter included in a clutch pedal-less MT vehicle. The paddle shifter is mounted on a steering wheel. The paddle shifter comprises an upshift switch and a downshift switch for determining an operating position. The upshift switch is pulled forward to emit an upshift signal 34u, and the downshift switch is pulled forward to emit a downshift signal 34d.

On the other hand, the lever-type pseudo shifter is a dummy that is different from the original shifter, similarly to the paddle-type shifter. The lever-type pseudo-shifter is structured to resemble a lever-type shifter provided in a clutch pedal-less MT vehicle. The pseudo-shifter of the lever type is configured to output an upshift signal 34u by tilting the shift lever forward, and output a downshift signal 34d by tilting the shift lever backward.

Battery electric vehicle 10E wheel 26 is provided with a wheel speed sensor 36. The wheel speed sensor 36 is used as a vehicle speed sensor for detecting the vehicle speed of battery electric vehicle 10E. Further, the electric motor 44 is provided with a rotation speed sensor 38 for detecting the rotation speed thereof.

Battery electric vehicle 10E includes a motor control device 50. The motor control device 50 is typically an electronic control unit (ECU) mounted on a battery electric vehicle 10E. The motor control device 50 may be a combination of a plurality of ECU. The motor control device 50 includes an interface, a memory, and a processor. An in-vehicle network is connected to the interface. The memories include a RAM for temporarily recording data, and a ROM for storing programs executable by the processor and various data related to the programs. The program is composed of a plurality of instructions. The processor reads and executes a program and data from a memory, and generates a control signal based on a signal acquired from each sensor.

For example, the motor control device 50 controls the electric motor 44 by PWM control of the inverter 42. Signals from the accelerator position sensor 32, the sequential shifter 24, the wheel speed sensor 36, and the rotation speed sensor 38 are input to the motor control device 50. When the sequential shifter 24 is a paddle type shifter, signals from an upshift switch and a downshift switch are input. The motor control device 50 processes these signals and calculates a motor torque command for controlling PWM of the inverter 42.

The motor control device 50 includes an automated mode (EV mode) and a manual mode (MT mode) as control modes. Automated mode is a normal control mode for operating a battery electric vehicle 10E as a common battery electric vehicle. The automatic mode is programmed to continuously change the output of the electric motor 44 in response to operation of the accelerator pedal 22. On the other hand, the manual mode is a control mode for driving battery electric vehicle 10E like MT vehicles. The manual mode is programmed to vary the output characteristics of the electric motor 44 for operation of the accelerator pedal 22 in response to upshift and downshift operations on the sequential shifter 24. This manual mode (MT mode) corresponds to “sequential shift mode”. The automatic mode and the manual mode can be switched.

The motor control device 50 comprises an automatic mode torque calculation unit 54 and a manual mode torque calculation unit 56. The units 54 and 56 may be independent ECU or ECU functions obtained by executing programs stored in memories on a processor.

The automatic mode torque calculation unit 54 has the function of calculating the motor torque when the electric motor 44 is controlled in the automatic mode. The automatic mode torque calculation unit 54 stores a motor torque command map. The motor torque command map is a map for determining the motor torque from the accelerator operation amount and the rotational speed of the electric motor 44. A signal of the accelerator position sensor 32 and a signal of the rotation speed sensor 38 are input to each parameter of the motor torque command map. The motor torque corresponding to these signals is output from the motor torque command map. Therefore, in the automatic mode, even if the driver operates the sequential shifter 24, the operation is not reflected in the motor torque.

The manual mode torque calculation unit 56 comprises a MT vehicle-model. Assuming that battery electric vehicle 10E is a MT vehicle, MT vehicle model is a model for calculating the drive wheel torque that should be obtained by operating the accelerator pedal 22 and the sequential shifter 24.

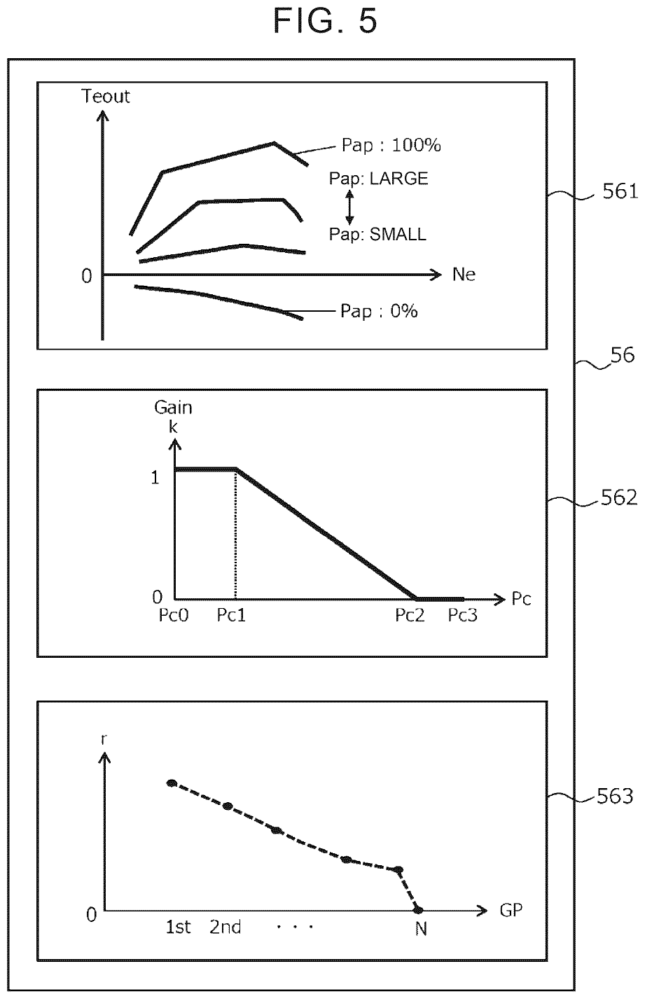

Referring to FIG. 5, a MT vehicle model included in the manual mode torque calculation unit 56 will be described. As shown in FIG. 5, MT vehicle model includes an engine model 561, a clutch model 562, and a transmission model 563. The engine, the clutch, and the transmission virtually realized by MT vehicle-model are referred to as a virtual engine, a virtual clutch, and a virtual transmission, respectively. In the engine model 561, a virtual engine is modeled. In the clutch model 562, a virtual clutch is modeled. In the transmission model 563, a virtual transmission is modeled.

The engine model 561 calculates a virtual engine rotation speed Ne and a virtual engine power torque Teout. The virtual engine rotation speed Ne is calculated based on the rotational speed Nw of the wheel, the overall reduction ratio R, and the slip-rate Rslip of the virtual clutch. For example, the virtual engine rotation speed Ne is expressed by the following equation (1).

$$Ne = Nw \times R / ( 1 - R slip ) \qquad Equation ( 1 )$$

The virtual engine output torque Teout is calculated from the virtual engine rotation speed Ne and the accelerator operation amount Pap. In calculating the virtual engine output torque Teout, as shown in FIG. 5, a map that defines the relation between the accelerator operation amount Pap, the virtual engine rotation speed Ne, and the virtual engine output torque Teout is used. In this map, the virtual engine output torque Teout with respect to the virtual engine rotation speed Ne is given for each accelerator operation amount Pap. The torque characteristic shown in FIG. 5 may be set to a characteristic assumed for a gasoline engine, or may be set to a characteristic assumed for a diesel engine. In addition, the torque characteristics may be set to characteristics assumed for a natural intake engine, or may be set to characteristics assumed for a supercharged engine.

The clutch model 562 calculates a torque transmission gain k. The torque transmission gain k is a gain for calculating a torque transmission degree of the virtual clutch according to the virtual clutch operation amount Pc. The virtual clutch operation amount Pc is typically 0% and is temporarily opened to 100% in conjunction with the switching of the virtual gear stages of the virtual transmission. The clutch model 562 has a map as shown in FIG. 5. In this map, the torque transmission gain k is given to the virtual clutch operation amount Pc. In FIG. 5, Pc0 corresponds to a position where the virtual clutch operation amount Pc is 0%, and Pc3 corresponds to a position where the virtual clutch operation amount Pc is 100%. The range from Pc0 to Pe1 and the range from Pc2 to Pc3 are dead zones in which the torque transmission gain k does not change depending on the virtual clutch operation amount Pc. The clutch model 562 calculates the clutch output torque Tcout using the torque transmission gain k. The clutch output torque Tcout is torque output from the virtual clutch. For example, the clutch output torque Tcout is given by the product of the virtual engine output torque Teout and the torque transfer gain k (Tcout=Teout×k).

In addition, the clutch model 562 calculates the slip-rate Rslip. The slip-rate Rslip is used to calculate the virtual engine rotation speed Ne in the engine model 561. The slip ratio Rslip can be calculated using a map in which the slip ratio Rslip is given to the virtual clutch operation amount Pc in the same manner as the torque transmission gain k.

The transmission model 563 calculates a gear ratio r. The gear ratio r is a gear ratio determined by the virtual gear stage GP in the virtual transmission. When the sequential shifter 24 is upshifted, the virtual gear stage GP is raised by one stage. On the other hand, the virtual gear stage GP is lowered by one stage in response to the downshift of the sequential shifter 24. The transmission model 563 has a map as shown in FIG. 5. In this map, the gear ratio r is given to the virtual gear stage GP such that the larger the virtual gear stage GP, the smaller the gear ratio r. The transmission model 563 calculates the transmission output torque Tgout using the gear ratio r and the clutch output torque Tcout obtained from the map. For example, the transmission output torque Tgout is given by the product of the clutch output torque Tcout and the gear ratio r (Tgout=Tcout×r). The transmission output-torque Tgout changes discontinuously in response to the change of the gear ratio r. This discontinuous change in the transmission output-torque Tgout causes a shift-shock, which results in the appearance of a vehicle equipped with a stepped transmission.

MT vehicle model calculates the drive wheel torque Tw using a predetermined reduction ratio rr. The reduction ratio rr is a fixed value determined by the mechanical structure from the virtual transmission to the drive wheels. The overall reduction ratio R described above is obtained by multiplying the reduction ratio rr by the gear ratio r. MT vehicle model calculates the drive wheel torque Tw from the transmission output torque Tgout and the reduction ratio rr. For example, the drive wheel torque Tw is given by the product of the transmission power torque Tgout and the reduction ratio rr (Tw=Tgout×rr).

The motor control device 50 converts the drive wheel torque Tw calculated by MT vehicle-model into a required motor torque Tm. The required motor torque Tm is the motor torque required to realize the drive wheel torque Tw calculated by MT vehicle model. In order to convert the drive wheel torque Tw into the required motor torque Tm, the reduction ratio from the output shaft of the electric motor 44 to the drive wheels is used. Then, the motor control device 50 controls the inverter 42 in accordance with the required motor torque Tm to control the electric motor 44.

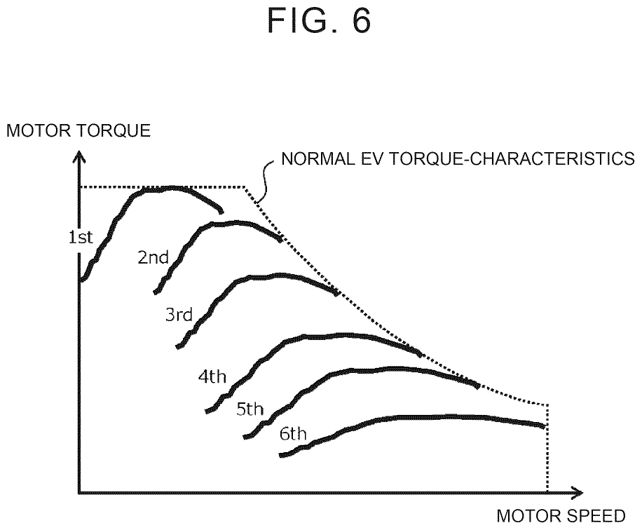

FIG. 6 is a diagram showing torque characteristics of an electric motor 44 realized by motor control using a MT vehicle-model compared with torque characteristics of an electric motor 44 realized by normal motor control as a battery electric vehicle (EV). According to the motor control using MT vehicle model, as shown in FIG. 6, a torque characteristic (solid line in the drawing) that simulates the torque characteristic of MT vehicle can be realized in accordance with the virtual gear stage set by the sequential shifter 24. In FIG. 6, the number of gear stages is six.

### 4-2. Second Configuration Example (Three-Pedal Mode)

FIG. 2 is a block-diagram illustrating a second configuration of the power control system of battery electric vehicle 10E according to 4B embodiment. Here, only a configuration different from the above-described first configuration example will be described. Therefore, in 4B of the drawings, the same configuration as the first configuration is omitted except for the motor control device 50.

Specifically, in the second configuration example, battery electric vehicle 10E includes the pseudo shift lever 27 and the pseudo clutch pedal 28 instead of the sequential shifter 24 provided in the first configuration example. The pseudo shift lever 27 is a pseudo shift device. The pseudo shift lever 27 and the pseudo clutch pedal 28 are dummies different from the original shift lever and the clutch pedal.

The pseudo shift lever 27 has a configuration simulating a shift lever included in MT of the vehicles. The arrangement and the operating feeling of the pseudo shift lever 27 are equivalent to those of the actual MT vehicles. The pseudo shift lever 27 is provided with, for example, positions corresponding to the gear stages of 1 speed, 2 speed, 3 speed, 4 speed, 5 speed, 6 speed, reverse speed, and neutral. The pseudo shift lever 27 is provided with a shift position sensor 27a that detects a gear stage by determining which position the pseudo shift lever 27 is in.

The pseudo clutch pedal 28 simulates a clutch pedal included in MT of the vehicles. The arrangement and operating feel of the pseudo clutch pedal 28 are equivalent to those of actual MT vehicles. The pseudo clutch pedal 28 is operated when the pseudo shift lever 27 is operated. That is, the driver depresses the pseudo clutch pedal 28 when it is desired to change the setting of the gear stage by the pseudo shift lever 27, and when the setting change of the gear stage is completed, the driver stops depressing and returns the pseudo clutch pedal 28 to its original state. The pseudo clutch pedal 28 is provided with a clutch position sensor 28a for detecting a depression amount of the pseudo clutch pedal 28.

The motor control device 50 receives signals from the accelerator position sensor 32, the shift position sensor 27a, the clutch position sensor 28a, the wheel speed sensor 36, and the rotation speed sensor 38. The motor control device 50 processes these signals and calculates a motor torque command for controlling PWM of the inverter 42.

As in the first configuration described above, the motor control device 50 includes an automated mode (EV mode) and a manual mode (MT mode) as the control mode. The automatic mode is programmed to continuously change the output of the electric motor 44 in response to operation of the accelerator pedal 22. On the other hand, the manual mode is a control mode for driving battery electric vehicle 10E like MT vehicles. The manual mode is programmed to change the output and output characteristics of the electric motor 44 relative to the operation of the accelerator pedal 22 in response to the operation of the pseudo clutch pedal 28 and the pseudo shift lever (pseudo shift device) 27. This manual mode (MT mode) corresponds to “Three-pedal mode”. The automatic mode and the manual mode can be switched.

The vehicle model included in the manual mode torque calculation unit 56 is similar to that shown in FIG. 5. However, the virtual clutch operation amount Pc is replaced with the depression amount of the pseudo clutch pedal 28 detected by the clutch position sensor 28a. The virtual gear stage GP is determined by the position of the pseudo shift lever 27 detected by the shift position sensor 27a.

### 5. Other Embodiments

As the pseudo-engine sound for increasing the tension of the driver, the tone of the pseudo-engine sound may be changed, and the pseudo-engine sound may include a favorite sound of the driver. In this case, in the articulation control, the control device 100 generates a mixed pseudo engine sound in which the pseudo engine sound and the driver's favorite sound included in the driver preference information 121 are mixed. Then, the control device 100 changes the tone of the mixed pseudo engine sound in accordance with the attribute of the sound desired by the driver. This makes it possible to further increase the tension of the driver as compared with the above-described embodiment.

What is claimed is:

1. A control device mounted on a battery electric vehicle that uses an electric motor as a power unit for travel and has a manual transmission (MT) mode in which a manual transmission vehicle is simulated, the control device comprising:
one or more storage devices that store preference information on preferences of a driver of the battery electric vehicle; and
one or more processors configured to generate a pseudo engine sound and output the pseudo engine sound through one or more in-vehicle speakers, wherein
the one or more processors are configured to:
acquire biometric information on the driver using a sensor mounted to the driver or a sensor mounted on the battery electric vehicle; and
perform tone control for changing a tone of the pseudo engine sound based on the preference information or the biometric information.

2. The control device according to claim 1, wherein:
the preference information includes a favorite sound of the driver; and
the tone control includes changing the tone of the pseudo engine sound according to an attribute of the favorite sound.

3. The control device according to claim 1, wherein:
the biometric information includes information on a heart rate; and
the tone control includes changing the tone of the pseudo engine sound according to fluctuations in the heart rate.

4. The control device according to claim 1, wherein the one or more processors are configured to adjust at least one of a sound pressure, a frequency, and a sound range of the pseudo engine sound in the tone control.

5. The control device according to claim 1, wherein:
the preference information includes a favorite sound of the driver; and
the one or more processors are configured to, in the tone control,
generate a mixed pseudo engine sound by mixing the pseudo engine sound and the favorite sound, and
change a tone of the mixed pseudo engine sound according to an attribute of the favorite sound.
